(19) United States
(12) Patent Application Publication
(10) Pub. No.: US 2013/0199734 A1
(43) Pub. Date: Aug. 8, 2013

(54) Method for manufacturing protein solutions and their concentration

(71) Applicants: Torsten SCHULTZ-FADEMRECHT, Maselheim (DE); Stefan BASSARAB, Biberach an der Riss (DE); Patrick GARIDEL, Norderstedt (DE)

(72) Inventors: Torsten SCHULTZ-FADEMRECHT, Maselheim (DE); Stefan BASSARAB, Biberach an der Riss (DE); Patrick GARIDEL, Norderstedt (DE)

(73) Assignee: BOEHRINGER INGELHEIM INTERNATIONAL GMBH, Ingelheim am Rhein (DE)

(21) Appl. No.: 13/565,186

(22) Filed: Aug. 2, 2012

(30) Foreign Application Priority Data
Aug. 19, 2011 (EP) 11 178 053.2

(51) Int. Cl. C07K 1/14

(52) U.S. Cl. CPC C07K 1/14
USPC 159/47.1

(57) ABSTRACT

The invention relates to a method for preparing highly concentrated liquid protein formulations. They are prepared by concentrating a protein solution by carrier gas drying at a reduced process pressure.

### BACKGROUND TO THE INVENTION

1. Technical Field

The invention relates to a method for manufacturing highly concentrated liquid protein formulations. The protein solutions are produced here by concentration by carrier gas drying at a reduced process pressure.

2. Background

For preparing highly concentrated protein solutions it is generally necessary, in a first step, to eliminate water from a protein-containing, correspondingly low-concentration solution. Depending on the degree of elimination, either highly concentrated solutions are formed or, if the water is eliminated almost completely, powder. This results in two possible ways of producing highly concentrated protein solutions. Either the protein solution is concentrated down until the target concentration is reached, or a protein-containing powder is reconstituted in a suitable liquid medium, according to the protein concentration required. Whereas the dewatering of smaller chemical molecules is generally unproblematic, in the case of biomolecules such as proteins the concentration or dewatering may cause damage to the molecule and hence a loss of activity. For dewatering solutions that contain biomolecules such as proteins, for example, there is a particular need to develop a process by which a solution of this kind, such as a protein solution, for example, can be concentrated while avoiding stress factors as far as possible.

The concentration of solutions by evaporation can most easily be carried out by simple air-drying under normal conditions/laboratory conditions. However, this method of drying is very time-consuming and therefore uneconomical. Because of the long drying time there is also the danger of protein damage. Suitable methodologies are therefore characterised in that they increase the evaporation rate substantially compared with “conventional” air-drying. As an alternative to evaporation, filtration processes, osmolytically driven processes and chromatographic methods are used to concentrate the proteins in solution (Shire et al., “Journal of Pharmaceutical Science, Vol. 93, No. 6, June 2004, Challenges in the Development of High Protein Concentration Formulations”).

An established method of concentrating protein solutions is filtration through semi-permeable membranes that retain the protein but allow buffer salts or adjuvants in general to pass through. However, filtration has the disadvantage that the protein that is to be processed may accumulate in large quantities on the membrane surface. This militates against the process efficiency and is therefore undesirable. Generally, a distinction is drawn between filtration processes with and without depletion of the protein concentration on the membrane surface. Examples of methods with depletion include tangential flow filtration and concentration using agitation cells. An example of filtration without depletion or without cleaning of the membrane is concentration in centrifuges, e.g. with Vivispin or Amicon centrifuge test tubes.

A major problem in filtration technology, particularly in the case of high viscosity solutions, is the blocking of the membranes, or fouling. In order to counteract these effects, in the case of tangential flow filtration, a high rate of overflow is set on the retentate side. In the case of the agitation cell the membrane surface is cleaned by an agitator. These techniques generate a shear effect on the protein which may be high at times, with the result that protein damage, particularly denaturing, may occur. Rosenberg et al. (Journal of Membrane Science 342: 50-59, “Ultrafiltration concentration of monoclonal antibody solutions: Development of an optimized method minimizing aggregation”) established during the tangential flow filtration of solutions containing antibodies, that there was increased turbidity as the shear stress increased. During the investigations, the proportion of insoluble protein aggregates rose with increasing transmembrane pressure or with increasing shear gradients in the channels of the filtration membrane used. Bee et al. (Biotechnol Bioeng. 2009 Aug. 1; 103(5): 936-943, “Response of a concentrated monoclonal antibody formulation to high shear”) and Maa et al. (Biotechnology and Bioengineering, Vol 54, No. 6, Jun. 20, 1997, “Protein denaturation by combined effect of shear and air-liquid interface”) also investigated the influence of shear forces on proteins. They found a synergistic effect between interface effects (air/protein solution) and shear gradients, which led to increased protein damage. Another method of concentrating protein solutions is ultrafiltration. However, the disadvantage of ultrafiltration is the formation of a protein-containing gel layer on the membrane surface. In particular, the filtration rate decreases rapidly as a result of the accumulation of protein on the membrane surface (fouling) (Rosenberg et al., Journal of Membrane Science 342: 50-59, “Ultrafiltration concentration of monoclonal antibody solutions: Development of an optimized method minimizing aggregation”). Huisman et al. (Journal of Membrane Science 179 (2000), 79-90, “The effect of protein-protein and protein-membrane interactions on the membrane fouling in ultrafiltration”) conducted investigations into the incidence of membrane fouling during the filtration of protein-containing solutions. According to the findings of Huisman et al. the collapse of the filtration rate can be put down to both the covering of the membrane surface with the protein that is to be filtered and also the build-up of a layer of protein on the membrane surface. The covering of the membrane surface with protein also results in polarisation or Donnan effects. These effects and the interactions between the protein and the dissolved adjuvants may lead to an accumulation or depletion of the adjuvants in the retentate, so that the composition of the final formulation is not correct (Stoner et al., Journal of Pharmaceutical Sciences, Vol. 93, No 9, September 2004, “Protein-Solute interactions affect the outcome of ultrafiltration/diafiltration operations”) or deviates from the theoretically predefined final formulation.

Another problem, particularly at high protein concentrations, is the dependency of the filtration rate on the viscosity of the protein solution that is to be filtered. The Darcy equation reflects this dependency. According to the equation:

$$\frac{d V}{d t \cdot F} = \frac{1}{R \cdot η} \cdot Δ p$$

dV/dt=filtrate volume flow
F=filter surface
R=filtration resistance
η=viscosity
Δp=transmembrane pressure drop

In accordance with the Darcy equation the filtration rate decreases with the viscosity. Other critical aspects are the heterogeneity of the pores of the filter materials, the protein binding to the membrane and the mechanical stability of the membranes. These above-mentioned aspects may adversely affect the process stability and the quality of concentration, particularly in the case of protein solutions.

There is therefore a need to provide an improved method of concentrating solutions that contain biomolecules such as protein solutions, in particular.

Patent application WO2009/073569 describes a method of preparing highly concentrated adjuvant-free solutions by filtration. In this method, the excipients contained in the solution are replaced with water by tangential flow filtration and then concentrated. Concentration may be carried out by tangential flow filtration or by centrifuging using suitable centrifuge test tubes (e.g. Vivaspin tubes). However, the disadvantages mentioned above still occur here, such as blocking of the membrane, a shift in the pH and high protein losses. In addition, it is difficult to carry out a membrane-based process of this kind aseptically.

Another method of concentrating protein solutions is the so-called “hanging drop” or “sitting drop” method. The driving force in this process is the vapour pressure difference between the protein solution and a second solution with a high salt content, the so-called reservoir. As the volumes used are in the lower μL range, this method is unsuitable for the production of highly concentrated pharmaceutical protein solutions. Another disadvantage of this method is the difficulty of controlling it as the colligative properties of the two solutions and hence the resulting vapour pressures change continuously. This method is only used for analytical purposes, for determining the crystallisation properties of proteins.

Another method of concentrating solutions is the use of convection driers. Here, a current of dry, warm or hot air is passed over the material to be dried, thus drying it. Both increasing the air temperature and carrying away the moisture accelerate the process of evaporation of the liquid. A commercially available system (TurboVap® evaporator) for concentrating solutions by evaporation is sold for example by the company Caliper Life Sciences GmbH. In this apparatus an air current the temperature of which can be controlled is passed helically into a container. The resulting chimney effect is supposed to assist with the discharge of the moist air, thus allowing drying to proceed more rapidly. This principle is suitable for both organic and aqueous solutions. However, the introduction of an air current into individual containers has the disadvantage that this requires very great technical expenditure to ensure that the same overflow speed and hence the same drying rate are present in each container. When hot air is used, protein damage may occur at the liquid/gas phase interface.

In contrast to convection drying, in contact drying the energy required for evaporating the water is passed over heated surfaces into the material that is to be dried. An additional possibility for accelerated concentration of a solution is lowering the process pressure in the drying chamber. Concentration at low process pressures or under a vacuum does increases the evaporation rate, on the one hand, but on the other hand the maximum achievable product temperature in the drying process is also limited, so that solutions with thermally unstable substances, in particular, can be satisfactorily concentrated by this process. Besides the conventional vacuum drying chambers made by the companies Nereus and Binder, for example, centrifugal evaporation (e.g. the mivac centrifugal evaporator EZ 2 series made by GeneVac) can also be used to avoid delays in boiling. The additional centrifuging of the solutions is also intended to prevent possible delays in boiling.

A disadvantage of convection dryers with closed vacuum chambers is that the moisture accumulates in the gas chamber so that the evaporation rate decreases over the process time and is difficult to control.

Another critical aspect in the concentration by evaporation of a protein solution stabilised with adjuvants is the simultaneous concentration of the adjuvants. This may lead to an unwanted additional increase in viscosity (Shire et al., Journal of Pharmaceutical Science, Vol. 93, No. 6, June 2004, “Challenges in the Development of High Protein Concentration Formulations”).

Another possible way of producing protein solutions is by dissolving protein-containing powders in a reconstitution medium. In these methods, protein-containing powders are prepared in a first step and are then reconstituted in a suitable medium in a second step. The highly concentrated protein solution is prepared by reconstituting the powder in substantially smaller volumes in relation to the initial starting solution and thereby concentrating the protein. The method of preparing the powders may be any desired method, provided that the protein is not damaged by the removal of the water and the adjuvants are suitable for the subsequent reconstitution. Methods of preparing powders described in the literature are freeze-drying, convection drying (spray-drying, warm air drying), vacuum drying and the production of protein-containing precipitates. Matheus et al. (JOURNAL OF PHARMACEUTICAL SCIENCES, VOL. 98, NO. 9, SEPTEMBER 2009, “Liquid High Concentration IgG1 Antibody Formulations by Precipitation”) describes the preparation of precipitates by salting out with ammonium sulphate or sodium citrate and using polymers (e.g. PEG 4000 or PEG 8000).

Precipitates may also be produced by methods using supercritical carbon dioxide (Winter et al.: J. Pharm. Science 85 (6): 586-594, “Precipitation of proteins in supercritical carbon dioxide”). Another possibility is ultrasound-induced precipitation. Another method described for the preparation of precipitates is precipitation using organic solvents (WO2008/132439). Allision et al. (ARCHIVES OF BIOCHEMISTRY AND BIOPHYSICS, Vol. 358, No. 1, October 1, pp. 171-181, 1998, “Effects of Drying Methods and Additives on Structure and Function of Actin: Mechanisms of Dehydration-Induced Damage and Its Inhibition”) dried a protein solution by passing dry nitrogen over the solution in a first step and then, in a subsequent step, allowing the sample to dry further in a lyophiliser in vacuo until a powder was formed. Stabenau (Dissertation “Drying and Stabilising of Proteins by Warm Air Drying and Application of Microdrops”, Ludwig-Maximilians University, Munich 2003) describes drying a protein solution by means of a warm air gassing device. In this “miniature single dose convection drying” warm air is passed over the solution in a vial. A disadvantage of this method is the considerable technical expenditure involved in concentrating single doses as each container is controlled separately through a gas nozzle. The material is dried at normal pressure and the gas is heated to increase the water evaporation rate. The disadvantage of this is that thermally unstable proteins, in particular, can be damaged by the introduction of heat.

Patent application WO 2004/060343A1 describes a method in which a solution containing antibodies is spray-dried and then reconstituted so as to form highly concentrated protein solutions. During this spray-drying the proteins are dehydrated down to powder form. To avoid damage during dehydration, additional adjuvants are generally needed which envelop the protein in the powder in an amorphous matrix. A disadvantage is that this thermodynamically unstable state of the amorphous matrix makes it necessary to protect the powder from any moisture in the air at all times as otherwise the amorphous matrix will crystallise and the protein may be damaged. Moreover it may be necessary after the reconstitution of the powders to eliminate the adjuvants by tangential flow, for example, and replace them by adjuvants that are more suitable for protein stabilisation in liquid form. This additional step involves increased technical expenditure and a greater risk of causing further damage to the protein by the additional procedure.

Mattern et al. (Pharm. Dev. Technol 4(2) (1997):199-208, 1997, “Formulation of proteins in vacuum dried glasses II. Process and storage stability in sugar-free amino acid systems) (European Journal of Pharmaceutics and Biopharmaceutic 44 (1997), 177-185, “Formulation of proteins in vacuum dried glasses I. Improved vacuum-drying of sugars using crystallising amino acids”) investigated the influence of adjuvants, particularly sugar and amino acids, on the vacuum drying of protein-containing solutions. The vacuum drying carried out by Mattern et al. was carried out in a freeze-dryer at 20° C. and 0.1 Pa. For the preparations, 1 ml of the protein-containing solution was transferred into 2 ml vials and dried for 24 hours under the conditions stated above. Willmann (Dissertation “Stabilisation of Pharmaceutical Protein Solutions by Vacuum-Drying”, Ludwig-Maximilians University, Munich 2003) investigated different vacuum technologies for the production of dry protein formulations. Willmann compared the technology and equipment shown in the following Table:

| Equipment/Company | Description of method |
|---|---|
| Rotary vacuum | The apparatus consists essentially of a |
| concentrator | centrifuge (1300 rpm), a cold trap |
| (Alpha-RVC) | (condenser temperature about −80° C.) |
| | and IR lamps (temperature: up to 60° C.) |
| | for heating the centrifuge chamber. |
| Vacuum drying chamber | The vacuum is produced by means of a |
| (made by Heraeus) | membrane vacuum pump. Temperature |
| | control is achieved by a jacket heater. |
| Vacuum drying chamber | This vacuum drying chamber, unlike the |
| (made by Memmert) | Heraeus vacuum drying chamber, contains |
| | a plate heater and no jacket heater. |
| IR-Dancer (IRD, made | The apparatus consists of an evaluatable |
| by Hettich-Zentrifugen) | chamber, a cold trap and a vacuum pump. |
| | The sample is heated by means of IR lamps. |
| | In addition the sample is set vibrating. |
| GT-Alpha 2-4 | Single chamber freeze-drying apparatus. |

Patent application US2006/0275306A1 describes a process which is characterised in that a powder produced by freeze-drying is reconstituted so as to obtain a stable isotonic protein solution with a protein concentration of at least 50 mg/ml. The protein concentration after reconstitution increases by comparison with the initial concentration before freeze-drying by a factor of 2-40. This patent also describes how during freeze-drying the protein is formulated with a lyoprotector in a molar ratio of 100-1500 mol of lyoprotector to 1 mol of antibody.

A major disadvantage of all the powder manufacturing technologies is the additional technical expenditure and hence the increase risk of protein damage. Another critical aspect is maintaining the sterility of the product. For example, when spray-drying bulk solution, aseptic conditions must be guaranteed both during drying and during the subsequent transfer into containers.

In drying processes for producing powders, as a rule further adjuvants have to be used which are capable of stabilising the protein during dehydration (Allison et al., ARCHIVES OF BIOCHEMISTRY AND BIOPHYSICS, Vol. 358, No. 1, October 1, pp. 171-181, 1998, “Effects of Drying Methods and Additives on Structure and Function of Actin: Mechanisms of Dehydration-Induced Damage and Its Inhibition”). As a rule disaccharides such as saccharose or trehalose are used for this, as they permit good protein stabilisation (Carpenter et al., Pharmaceutical Research, Vol. 14, No. 8, 1997, “Rational design of stable lyophilized protein formulations: some practical advice”). Not only the adjuvant used but also the proportion of adjuvant in the powder and hence the ratio of protein to adjuvant is important for good protein stabilisation. If the protein/adjuvant ratio is unfavourable, the protein may be damaged (Chang et al., Pharmaceutical Research, Vol. 13, No. 2, 1996, “Development of a stable freeze dried formulation of recombinant human IL1-Receptor antagonist”). For producing highly concentrated protein solutions there is also the disadvantage that the addition of adjuvants such as disaccharides, for example, additionally increases the viscosity after the dissolving of the powder. A further disadvantage is that the speed of reconstitution may be impaired by the high concentrations of protein and adjuvants (Shire et al., Journal of Pharmaceutical Science, Vol. 93, No. 6, June 2004, Challenges in the Development of High Protein Concentration Formulations).

The vacuum drying of protein-containing solutions to the point of powder formation has the disadvantage that the so-called rubber state passes very slowly, particularly when typical adjuvants such as disaccharides are present. The drying properties in this rubber state are inadequate with the result that the residual moisture of the powder obtained is very high, which is generally associated with additional instability during storage of the protein (Mattern et al., Pharm. Dev. Technol 4(2) (1997):199-208, 1997, European Journal of Pharmaceutics and Biopharmaceutic 44 (1997), 177-185). Only by adding crystallisable additives such as phenylalanine were Mattern et al. able to prepare powders with a low residual moisture content. In mechanistic terms the improved drying property is explained by the fact that the amorphous protein-containing matrix consisting for example of an amorphous sugar and the protein is deposited on the crystalline phenylalanine and is able to dry more easily thanks to the resulting increased surface area. Disadvantages to the preparation of highly concentrated protein solutions using this method are the poor solubility of phenylalanine and the long reconstitution time of the powder determined by the solubility.

There is therefore a strong need to provide an improved, gentle method of concentrating protein solutions, particularly in aseptic manufacture in the pharmaceutical sector.

### SUMMARY OF THE INVENTION

The invention describes a method of concentrating a protein solution comprising the following steps:
a. Preparing a protein solution,
b. Optionally transferring the protein solution from (a) into individual containers,
c. Transferring the protein solution from (a) or the individual containers from (b) into an apparatus comprising the following components:
   i. process chamber (7),
   ii. vacuum pump (8),
   iii. gas connection (1),
   iv. at least one inlet (5),
   v. at least one outlet (6),
   vi. flow sensor (3),
   vii. pressure sensor (4),
   viii. at least two valves (2),
   ix. optionally a perforated plate (10),
   x. optionally a recirculating pump (11) for protein solution (9) and bypass with connections (12) and (13),
d. Applying a gas current (also referred to as a carrier gas current) to the apparatus from step c), wherein
   i. the process pressure is reduced,
   ii. the flow rate of the gas (also referred to as carrier gas) is uniform,
e. Removing the concentrated protein solution or the individual containers from the apparatus according to method step c).

Thus the present invention provides a particularly gentle and virtually loss-free method for concentrating preferably adjuvant-free protein solutions. The process may be carried out in individual containers or in a primary packaging means or as bulk goods in a vat or pipe. The concentration is carried out using a carrier gas at reduced process pressures.

An essential feature of the present invention is the very gentle concentration of protein solutions by a combined process consisting of carrier gas drying at reduced process pressures. Particularly by comparison with tangential flow filtration, which is to be regarded as the standard method up to now for concentrating protein solutions, there are no additional shear forces during the concentration process and the interface effects are greatly reduced. The tangential flow filtration used as standard generally serves two processes:
ultrafiltration, which is used solely for concentration,
diafiltration, which serves to produce concentration with buffering.

The advantage of the method according to the invention, by contrast, lies particularly in the following aspects:
Carrier gas drying operates without losses, particularly during concentration in the primary packaging (for example the vial).
The method according to the invention can be carried out without any problems when used in the primary packaging means for small amounts. The minimum amount to be used is defined from the initial concentration of the protein solution, the desired concentration factor and the desired target volume of the protein solution. There are therefore no technically defined minimum quantities as determined for example by the dead volume of an apparatus.
By avoiding semi-permeable membranes as used for example in tangential flow filtration and centrifugation processes, no unwanted effects occur that greatly reduce the process efficiency and have an adverse effect on the composition of the formulation. These negative influences that occur when semi-permeable membranes are used include for example polarisation and Donnan effects and micro-fouling.
In the method according to the invention there is no need for any high flow speeds or shear gradients of the kind that occur with tangential flow, for example. This results in a process that is very gentle to the protein.
Thanks to the uniform concentration rate over the entire concentration process the method according to the invention is easy to control and monitor. In-process control to achieve the concentration level may for example be obtained by weighing the individual containers. A uniform concentration rate is not achieved in tangential flow filtration, for example, by contrast with the method according to the invention. There, the concentration rate decreases sharply over time, for example as a result of the increasing viscosity, due to the blocking of the membrane.
Thanks to the technically simple construction, concentration can easily be achieved under aseptic conditions.

These advantages are demonstrated by the Examples according to the invention.

FIG. 10 (Example 5) shows the protein concentrations at different times. For this, the gravimetrically determined protein concentrations were compared with the protein concentrations obtained by UV-spectroscopy. The good conformity between the protein concentration determined gravimetrically and that determined by UV spectroscopy shows that the concentration takes place without any significant protein losses.

Example 6 also shows that formulation screening is possible with minimal amounts. In this Example, concentration was carried out with a starting volume of protein solution of 1 ml of each formulation to be tested. 250 μl of protein solution were used for the re-dilution. The use of such small amounts/volumes of protein solutions is particularly important for applications in formulation screening.

As the method according to the invention does not require the use of membranes, polarisation or Donnan effects are avoided. In methods using membranes, the coating of the membrane surface with protein leads to polarisation or Donnan effects. As a result of these effects and the interactions between protein and dissolved adjuvants, concentration or depletion of the adjuvants in the retentate may occur, with the result that the composition of the final formulation is not correct or differs from the final formulation predicted in theory.

The method according to the invention also has the technical advantage that the water vapour concentrated in the gas phase during the concentration process is dynamically expelled from the process chamber and hence the water evaporation rate is both rapid and also constant over the process time.

The water evaporation rate can be additionally increased or controlled by applying a vacuum.

Another feature of the present invention is the temperature control of the carrier gas. By the choice of process temperature, the temperature is adjusted so as to achieve the highest possible water evaporation rate by means of the highest possible process temperature without having any negative effects on the integrity of the protein. This results in the technical advantage that ice formation is avoided even at a very high water evaporation rate.

Another technical advantage, particularly with respect to tangential flow filtration, is the substantially loss-free concentration process. This is a result of the fact that the concentration process can take place directly in the final primary packaging means. By contrast, liquid methods such as the previously mentioned tangential flow filtration, as well as centrifugation methods using membrane-containing centrifuge tubes (e.g. Vivispin or Amicon), have high protein losses in some cases as a result of the dead volume of the apparatus and the membranes. Additional losses result from protein adsorption on the surfaces in contact with the product, particularly the membranes.

The method according to the invention has the further advantage over the prior art that the novel method can easily be controlled with minimal technical expenditure. This advantage is essentially based on the fact that the concentration of the protein solution over the process as a whole does not exhibit any significant change in the concentration rate or the water evaporation rate. Over the preparation process there are no dependencies between the concentration rate or water evaporation rate and the chemical and physicochemical properties of the solution, such as for example the pH of the solution, the ion intensity and the isoelectric point of the protein. Moreover, the concentration rate is independent of the viscosity of the solution and even after the formation of gel-like states it does not show any significant changes in the water evaporation rate compared with low viscosity water-like solutions.

Moreover, there are no uncontrolled changes in the protein/adjuvant ratio. These may occur for example in methods that concentrate or dialyse proteins through semi-permeable membranes (Stoner et al., Journal of Pharmaceutical Science, Vol 93, No. 9, 2004, “Protein-Solute Interactions effect the outcome of ultrafiltration/diafiltration operations”). On these membranes so-called Donnan potentials are formed which interfere with the free exchange or the elimination of adjuvants. As a result there may be an accumulation or depletion of adjuvants compared with the desired adjuvant concentrations, which in turn may lead to an impairment of the protein stability and a change in osmolarity.

Methods that produce highly concentrated protein solutions by dissolving protein-containing powders generally require additional adjuvants which usually have to be eliminated again after the reconstitution of the protein. These adjuvants stabilise the proteins during the powder production and have the function of preventing protein damage. Powder manufacturing processes give rise to dehydration stress as a result of the almost total removal of water for the protein. As a result, changes in the configuration of the protein in the powder take place which may lead to irreversible protein damage after the reconstitution.

There are other stress factors for the protein, specific to the method. Lyophilisation gives rise to an additional freezing stress. During freezing or during ice crystal formation, comparatively hydrophobic interfaces are formed by the ice which may have a damaging effect on the protein that is still in solution. Selective precipitation of adjuvants during freezing may lead to adverse pH shifts. Spray-drying causes shear stress during the nebulisation of the solutions. The nebulising of protein solutions increases the phase interface between the protein solution and the gas phase. As a result of the increased interface, more denaturing of the protein may occur. Furthermore, the subsequent drying produces thermal stress on the protein.

It is known that damage to the protein can be suppressed by a suitable choice of adjuvants. The prior art uses sugar or amino acids. The success of the stabilisation also depends crucially on the thermodynamic state of the adjuvants in the dried state. Crystalline adjuvants cannot stabilise proteins as they do not counteract the dehydration stress. Amorphous structures are needed for this. Sugars, in particular, can very easily be made amorphous by lyophilisation and spray drying. However, stabilisation of the proteins in an amorphous matrix has the disadvantage that the amorphous structures of these adjuvants are unstable and have a tendency to crystallise. In this process, irreversible formation of protein aggregates generally occurs. The advantage of the present invention is that no additional adjuvants are required which have to be removed subsequently during the later preparation of the pharmaceutical formulation. As a result of the very gentle process it is even possible to concentrate protein solutions that are free from adjuvants.

### DESCRIPTION OF THE FIGURES

FIG. 1: Schematic representation of the drying apparatus

In this embodiment of a drying apparatus the protein solution to be concentrated is first transferred into individual containers, e.g. a primary packaging means (9). The ampoules are not shown to scale. The use of individual containers is not restricted to a particular type or size of ampoules such as for example injection ampoules or injection vials. Both glass and plastic ampoules may be used. It is also possible to use carpules or dishes of any kind, in addition to ampoules. To avoid surface effects, it is also possible for example to use primary packaging means with modified, particularly water-repellent, glass surfaces. These include for example vials coated with silicon dioxide or silicon oil. It is also possible to use vials which have been passivated by coating with hexamethyldisiloxane.

The carrier gas is passed from the gas connection (1) through the chamber, by means of a vacuum pump (8) at the outlet (6) from the process chamber (7). The inlet (5) of the carrier gas is located directly adjacent to the base of the chamber. The carrier gas is passed through a perforated plate (10) to the outlet in the upper region of the chamber. The gas rate and the absolute pressure are controlled by means of two sensors (flow sensor (3) and pressure sensor (4)) and by two needle valves (2). The chamber volume is not fixed and is dependent on the number and size of the individual containers. The carrier gas rate should be adjusted in accordance with the chamber volume and overall evaporation rate (sum of the evaporation rates per individual container) so that the humidity during the concentration process does not exceed 5% and the gas flow is from laminar to slightly turbulent through the chamber. Turbulence on the individual containers may adversely affect the evaporation rate.

The supply of the carrier gas (5) is schematically shown in the drawings by a single connection to the chamber. Other embodiments with additional connections ideally arranged symmetrically around the chamber are possible. For example, two connections may be arranged at an angle of 180° to one another. The overall carrier gas rate in this case will be divided into equal parts on the two connections. The advantage of additional carrier gas inlets is the more homogeneous flow of gas through the process chamber (7). In another embodiment the inlets may be uniformly distributed and connected directly to the base of the chamber.

Analogously to the inlets, additional outlet connections may be provided for optimising the gas flow. For a uniform evaporation rate it is important to provide a uniform flow rate in the process chamber. Both an excessively low flow rate and too high a flow rate may reduce the evaporation rate. In the first case there is an increase in the humidity of the carrier gas over the individual container and, consequently, a reduced evaporation rate. In the second case, the occurrence of turbulence, particularly on the individual container, may cause re-mixing and, consequently, a less favourable removal of the water vapour.

FIG. 2:

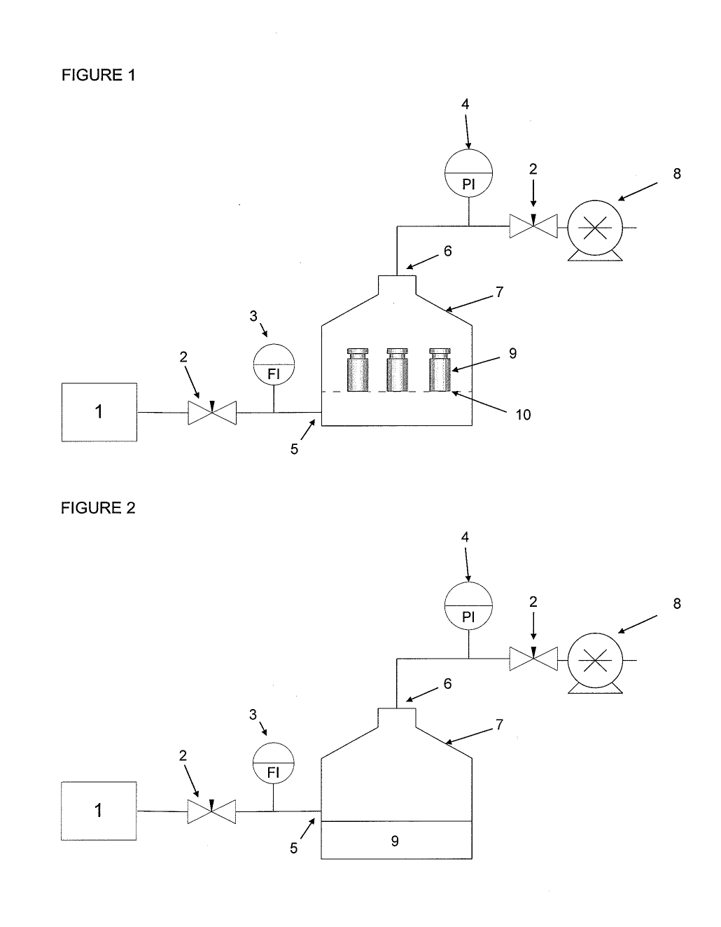

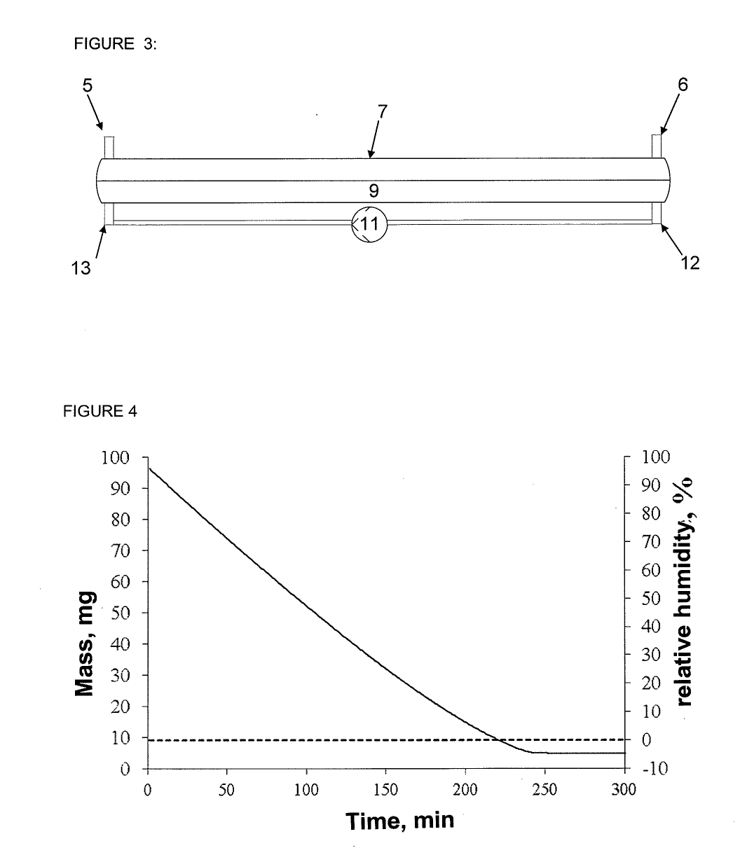

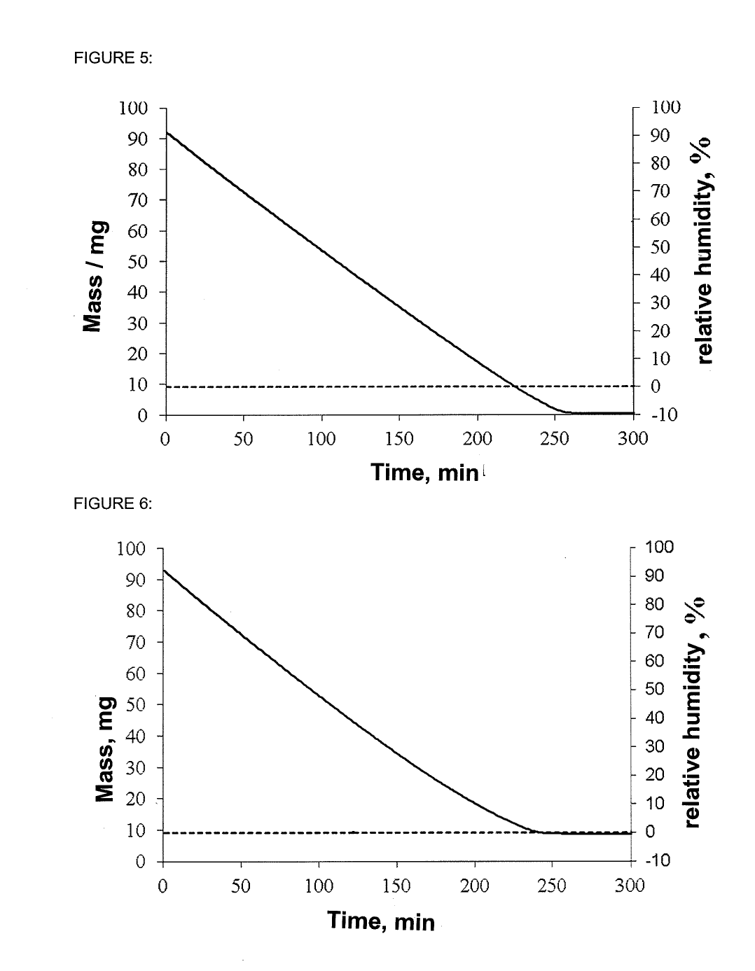

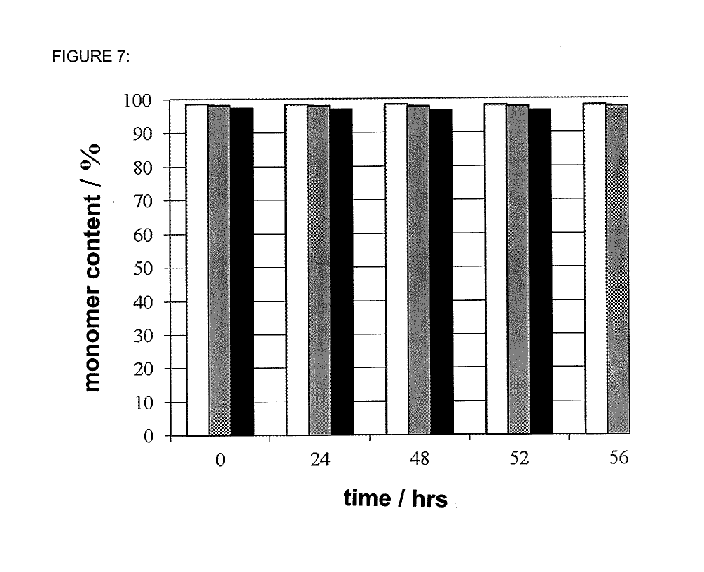

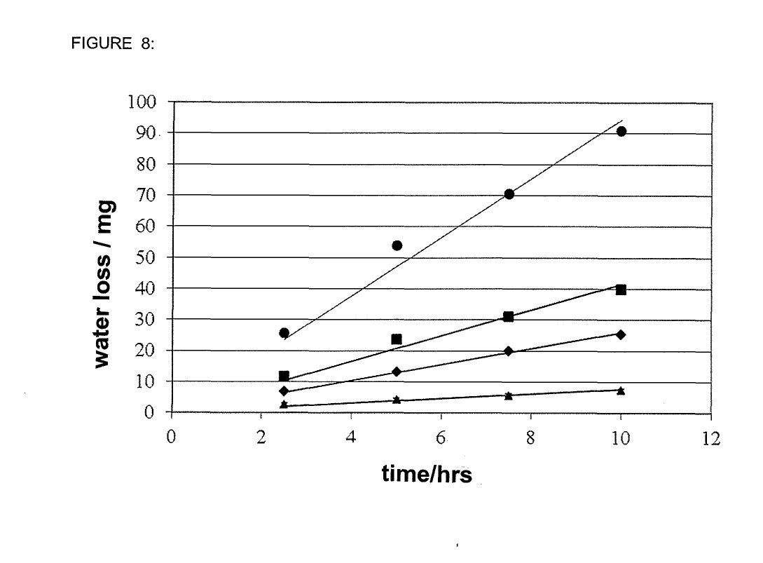

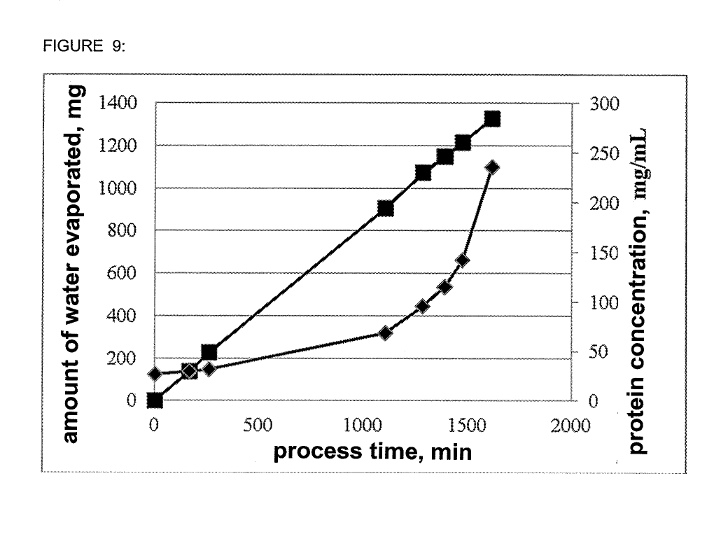

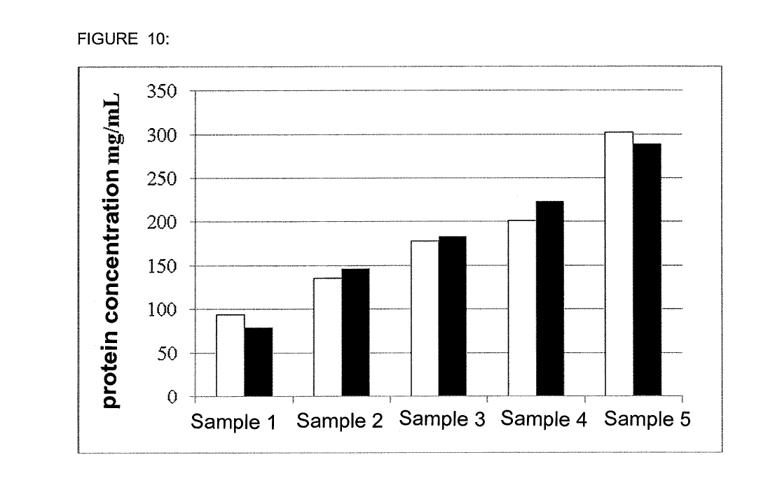

FIG. 2 shows another embodiment of a drying apparatus. Here the protein solution (9) is concentrated not in the individual container but in bulk form. The solution is contained directly in the process chamber (7) or in a vat. The connection for the carrier gas (5) is located above the liquid level and is passed over the solution. The outlet (6) can be provided on the lid of the chamber or vat, as described in FIG. 1. Analogously to the description of FIG. 1, in another embodiment other inlets and outlets may be integrated in the apparatus. The shape of the chamber is not restricted to the vat shape.

FIG. 3:

FIG. 3 shows another possible geometry of the process chamber. In this tubular chamber (7) the carrier gas is conveyed to the outlet (6) via the inlet (5). The protein solution (9) is circulated and thereby homogenised through a bypass by means of the pump (11). The direction of flow of the protein solution in the bypass may be both from the connection (12) to the connection (13) and in the reverse direction. The regulation of the carrier gas rate and the absolute pressure in the tube are carried out as shown in the description of FIGS. 1 and 2.

FIG. 4:

Representation of the loss of mass of an aqueous protein solution (IgG1a). The loss of mass over time was determined in the dynamic sorption balance at a gas rate of 200 cm³/min and a relative humidity of 0% r.h. The initial protein concentration was 51 mg/ml. The solids content of the solution was 5% (w/v). Continuous line: Pattern of the mass of the liquid over the process time, broken line: percentage relative humidity in the measuring chamber.

FIG. 5:

Representation of the loss of mass of an aqueous protein solution (IgG1b). The loss of mass over time was determined in the dynamic sorption balance at a gas rate of 200 cm³/min and a relative humidity of 0% r.h. The initial protein concentration was 5.1 mg/ml. The solids content of the solution was 0.5% (w/v). Continuous line: Pattern of the mass of the liquid over the process time, broken line: percentage relative humidity in the measuring chamber.

FIG. 6:

Representation of the loss of mass of an aqueous protein solution (IgG2a). The loss of mass over time was determined in the dynamic sorption balance at a gas rate of 200 cm³/min and a relative humidity of 0% r.h. The initial protein concentration was 87 mg/ml. The solids content of the solution was 9% (w/v). Continuous line: Pattern of the mass of the liquid over the process time, broken line: percentage relative humidity in the measuring chamber.

FIG. 7:

Representation of the monomer contents during concentration as a function of the process time.

White bar: monomer contents IgG1c
Grey bar: monomer contents IgG1d
Black bar: monomer contents IgG1b

FIG. 8:

Representation of the loss of water or mass from the individual glass test tubes as a function of the process time and process conditions.

Circular symbols: process pressure: 200 mbar/carrier gas rate: 4 L/min

Rectangular symbols: process pressure: 600 mbar/carrier gas rate: 4 L/min

Diamond shaped symbols: process pressure: 1013 mbar/carrier gas rate: 4 L/min

Triangular symbols: process pressure: 600 mbar/carrier gas rate: 0 L/min

FIG. 9:

Representation of the concentration of mass IgG4a. The quantity of water evaporated is plotted on the left hand vertical axis. The corresponding protein concentration is shown on the right hand vertical axis. In the diagram the changes in the quantities of water over time are indicated by rectangular symbols and the protein concentrations by diamond-shaped symbols. From the data of the amounts of water a linear regression was carried out, the gradient of which makes it possible to determine the water evaporation rate.

FIG. 10:

In FIG. 10 the protein concentrations measured by UV-spectroscopy are compared with the protein concentrations determined gravimetrically. The protein concentration determined gravimetrically is obtained from the initial concentration and the loss of mass of the individual container at the time of sampling. As the solution does not contain any vaporisable substances other than water, the loss of mass is equated with the quantity of water evaporated. The protein concentration can be calculated in mg/ml by including the density of water.

White bar: Protein concentration determined gravimetrically
Black bar: Protein concentration determined by UV spectroscopy

### DETAILED DESCRIPTION OF THE INVENTION

The highly concentrated liquid protein formulations are prepared according to the present invention by concentrating a protein solution by carrier gas drying at a reduced process pressure. FIG. 1 shows a schematic representation of the drying apparatus. In this embodiment the protein-containing solution that is to be concentrated is transferred into a primary packaging means, in this case test tubes, and placed in a drying chamber. The test tubes are arranged on a perforated plate. The dry carrier gas is supplied to the chamber (e.g. at the base) and flows over the perforated plate, past the vial, thus absorbing moisture. At the upper part of the chamber the carrier gas charged with moisture is extracted. The carrier gas is either dried air or, preferably, dry nitrogen. The process pressure or the vacuum is adjusted for example by means of a vacuum pump and a suitable needle valve. The flow rate of the carrier gas is also adjusted by means of a regulator valve. This regulator valve is located in front of the drying chamber in the direction of flow. Both the flow rate and also the process pressure are measured by measuring devices. The concentration of the solutions is not restricted to particular primary packaging means. Any desired containers may be used, such as for example bottles, ampoules or carpules. In addition to concentration in single dose containers, corresponding bulk drying is also possible. For this purpose, suitable dishes or other containers are used.

In another embodiment the drying chamber is directly filled with the protein-containing solution and concentrated. The carrier gas is fed in above the surface of the liquid. However, alternatively, it may also be fed in at the base of the chamber.

In another embodiment the protein solution is pre-concentrated, for example by tangential flow filtration, and then adjusted to the target concentration using the drying apparatus described above. The advantage of this procedure is that the entire process can be speeded up. The viscosity of the protein solution as a limiting factor of tangential flow filtration plays a minor part in this procedure.

In another embodiment, in a first process step the protein solution is buffered against water and pre-concentrated. The aqueous protein solution is then concentrated in the drying chamber described above. In order to adjust the composition of the solution the aqueous protein solution is concentrated for example to 1.5 to 2.0 times the target concentration and in a subsequent step it is re-diluted with multiply concentrated adjuvant solutions to the target protein concentration and desired composition of the formulation. A particular advantage of this procedure is the possibility of preparing and testing different protein formulations at minimal cost. The addition of the adjuvants in metered amounts after the concentration process also has numerous advantages. The adjuvant concentrations can be adjusted precisely. The adjuvants do not interfere with the drying process, for example by additionally (undesirably) increasing the viscosity. The course of the drying can be monitored by weighing the container, if single dose containers are used. The loss of mass gives the actual protein concentration. In the case of bulk drying and also when using titre plates, the actual protein concentration can be determined by specific analytical measurements of concentration (e.g. UV spectroscopy).

### DEFINITIONS

Terms and names used within the scope of this description of the invention have the following meanings defined hereinafter. The details of weights and percentages by weight, unless otherwise stated, are based on the dry mass of the compositions or the solids content of the solutions/suspensions. The general terms “containing” or “contain” include the more specialised embodiment “consisting of”. Moreover, “singular” and “plural” are not used in a restrictive capacity.

Polarisation effect: The term polarisation effect in membrane technology denotes an effect which is produced on the membrane surface by a concentration polarisation. The concentration polarisation is formed during concentration through a semi-permeable membrane by the formation of a covering layer chiefly consisting of macromolecules that cannot pass through the membrane. If the covering layer changes into a gel-like state as a result of the high protein concentration, the flux rate generally decreases, for example in is tangential flow filtration. Unlike fouling, the concentration polarisation is reversible. This means that once the covering layer has been removed, for example by rinsing the membrane surface, the original flux rate can be achieved again.

Fouling: The term fouling in membrane technology refers to the depositing of dissolved substances as well as macromolecules such as proteins, for example, in the pores of the membrane material. Membrane fouling is partly irreversible and cannot be totally reversed by rinsing the membrane.

Flux rate: The flux rate is a measurement of the filtration performance in membrane-bound filtration processes. The flux rate is referred to as the filtration speed either absolutely in units by volume per unit of time or standardised to the filter area present.

Donnan effects: The Donnan effect denotes an effect caused by the formation of a Donnan potential. Donnan potentials may be produced during filtration processes on semi-permeable membranes if the non-permeable macromolecule is also a charge carrier, for example in the case of proteins. Because of the need for the solutions to be electroneutral, charges of the macromolecule lead to an uneven distribution of small membrane-bound ions on both sides of the membrane. This uneven distribution of ions causes a Donnan potential to build up. This is generally associated with a disruption of the osmotic pressure through the membrane.

Colligative properties: The term colligative properties denotes a property of a substance which depends only on the number of particles but not on the nature of the particles. An example of this is the lowering of vapour pressure in aqueous solutions caused by the dissolved particles.

Rubber state: The rubber state denotes a state in amorphous substances. Amorphous states are characterised in that there are no crystalline structures present. The so-called glass transition temperature is characteristic of amorphous substances. Below this temperature the particles are immobilised in the amorphous matrix and have only very slight mobility. When the glass transition temperature is reached the mobility of the particles rapidly increases, and this is linked for example to a reduction in the viscosity of the substance. After exceeding the glass transition temperature the substance is changed into the so-called rubber state. In drying technology the rubber state is characterised in that the drying efficiency decreases and in some cases only dried goods with a high residual moisture content are obtained.

Bulk/Bulk drying: The term bulk denotes, particularly in pharmaceutical biotechnology, a product that is not packaged in primary packaging means, such as for example protein solutions. The term bulk drying is derived from the fact that for example a protein solution is not dried in the primary packaging means (e.g. test tube, ampoule, carpule) but as a bulk product in correspondingly larger containers such as dishes, for example.

Uniform: The term uniform in connection with the present invention relates to the gas flow through the process chamber which is directed in one direction of flow. For example, the uniform gas flow may be directed from the bottom of the process chamber towards the top of the process chamber. Ideally, the uniform flow in the process chamber is at the same speed and in the same direction at every point in the chamber. Another characteristic of the uniform flow is the substantially laminar flow around individual containers such as vials, for example. Turbulence may adversely affect the elimination of the water vapour by the gas, as a result of backflow. The uniform flow rules out special forms of gas flow such as helical flow profiles, for example.

Clean air: The term clean air denotes air with low concentrations of admixed substances. Clean air is characterised in that it is suitable for aseptic processes for producing sterile products.

Loss-free: The term loss-free relates to the recovery rate of the active substance used in the manufacturing process. Loss-free indicates a process with an active substance recovery of ≧95%. Loss-free also means loss-free within the scope of the accuracy of analysis. This is about +/−5%, e.g. in UV measurement of the protein solution or measurement of the mass weight.

The term “active substances” denotes substances which produce an effect or a reaction in an organism. If an active substance is used for therapeutic purposes in a person or on an animal body it is referred to as a drug or medicament.

Examples of active substances are insulin, insulin-like growth factor, human growth hormone (hGH) and other growth factors, tissue plasminogen activator (tPA), erythropoietin (EPO), cytokines, e.g. interleukins (IL) such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN)-alpha, -beta, -gamma, -omega or -tau, tumour necrosis factor (TNF) such as TNF-alpha, -beta or -gamma, TRAIL, G-CSF, GM-CSF, M-CSF, MCP-1 and VEGF. Other examples are monoclonal, polyclonal, multispecific and single chain antibodies and fragments thereof such as for example Fab, Fab′, F(ab′)₂, Fc and Fc′ fragments, light (L) and heavy (H) immunoglobulin chains and the constant, variable or hypervariable regions thereof as well as Fv and Fd fragments (Chamov et al., 1999). The antibodies may be of human or non-human origin. Humanised and chimeric antibodies are also possible. This also relates to conjugated proteins and antibodies which are connected for example to a radioactive substance or a chemically defined pharmaceutical substance.

Fab fragments (fragment antigen binding=Fab) consist of the variable regions of both chains which are held together by the adjacent constant regions. They may be produced for example from conventional antibodies by treating with a protease such as papain or by DNA cloning. Other antibody fragments are F(ab′)2 fragments which can be produced by proteolytic digestion with pepsin.

By gene cloning it is also possible to prepare shortened antibody fragments which consist only of the variable regions of the heavy (VH) and light chain (VL). These are known as Fv fragments (fragment variable=fragment of the variable part). As covalent binding via the cysteine groups of the constant chains is not possible in these Fv fragments, they are often stabilised by some other method. For this purpose the variable regions of the heavy and light chains are often joined together by means of a short peptide fragment of about 10 to 30 amino acids, preferably 15 amino acids. This produces a single polypeptide chain in which VH and VL are joined together by a peptide linker. Such antibody fragments are also referred to as single chain Fv fragments (scFv). Examples of scFv antibodies are known and have been described, cf. for example Huston et al., 1988.

In past years various strategies have been developed for producing multimeric scFv derivatives. The intention is to produce recombinant antibodies with improved pharmacokinetic properties and increased binding avidity. In order to achieve the multimerisation of the scFv fragments they are produced as fusion proteins with multimerisation domains. The multimerisation domains may be, for example, the CH3 region of an IgG or helix structures (“coiled coil structures”) such as the Leucine Zipper domains. In other strategies the interactions between the VH and VL regions of the scFv fragment are used for multimerisation (e.g. dia-, tri- and pentabodies).

The term diabody is used in the art to denote a bivalent homodimeric scFv derivative. Shortening the peptide linker in the scFv molecule to 5 to 10 amino acids results in the formation of homodimers by superimposing VH/VL chains. The diabodies may additionally be stabilised by inserted disulphide bridges. Examples of diabodies can be found in the literature, e.g. in Perisic et al., 1994.

The term minibody is used in the art to denote a bivalent homodimeric scFv derivative. It consists of a fusion protein which contains the CH3 region of an immunoglobulin, preferably IgG, most preferably IgG1, as dimerisation region. This connects the scFv fragments by means of a hinge region, also of IgG, and a linker region. Examples of such minibodies are described by Hu et al., 1996.

The term triabody is used in the art to denote a trivalent homotrimeric scFv derivative (Kortt et al., 1997). The direct fusion of VH-VL without the use of a linker sequence leads to the formation of trimers.

The fragments known in the art as mini antibodies which have a bi-, tri- or tetravalent structure are also derivatives of scFv fragments. The multimerisation is achieved by means of di-, tri- or tetrameric coiled coil structures (Pack et al., 1993 and 1995; Lovejoy et al., 1993).

The term “adjuvants” refers to substances that are added to a formulation, in the present invention a powder, particularly a spray-dried powder. Adjuvants normally do not have any pharmaceutical activities themselves and are used to improve the formulation of the actual active substance or to improve a particular aspect of it (e.g. storage stability).

A pharmaceutical “adjuvant” denotes a part of a drug or a pharmaceutical composition and ensures, among other things, that the active substance reaches the site of activity and is released there. Adjuvants have three basic functions: a carrier function, controlling the release of active substance and increasing stability. Adjuvants are also used to prepare pharmaceutical forms that are changed in their duration or speed of effect as a result.

The term “amino acid” refers to compounds that contain at least one amino and at least one carboxyl group. Although the amino acid is normally in the α-position relative to the carboxyl group any other arrangement in the molecule is also possible. The amino acid may also contain other functional groups such as, for example, amino, carboxamide, carboxyl, imidazole, thio groups and other groups. Amino groups of natural or synthetic origin, racemic or optically active (D- or L-) including various stereo isomeric ratios are used. For example, the term isoleucin includes both D-isoleucin, L-isoleucin, racemic isoleucin and various proportions of the two enantiomers.

The terms “peptide”, “polypeptide” or “protein” refer to polymers of amino acids consisting of more than two amino acid groups.

The term peptide, polypeptide or protein is used as a pseudonym and includes both homo- and heteropeptides, that is, polymers of amino acids consisting of identical or different amino acid groups. A “di-peptide” is thus synthesised from two peptidically linked amino acids while a “tri-peptide” is formed from three peptidically connected amino acids. The term “protein” used here refers in particular to polymers of amino acids with more than 20 and, in particular, more than 100 amino acid groups.

The term “small protein” denotes proteins under 50 kD or under 30 kD or between 5-50 kD. The term “small protein” also denotes polymers of amino acid groups with less than 500 amino acid groups or less then 300 amino acid groups or polymers with 50-500 amino acid groups. Preferred small proteins include, for example, growth factors such as “human growth hormone/factor”, insulin, calcitonin or the like.

The term “protein stability” denotes a monomer content of more than 90%, preferably more than 95%.

The term “oligosaccharide” or “polysaccharide” denotes multiple sugars which are synthesised from at lest three monomeric sugar molecules.

The term “dosage” or “dosages” refers to the quantity of the substance, particularly a therapeutic active substance, which is delivered when an applicator is used. The critical factor for the dose is the proportion of substance, particularly active substance, in the protein solution.

The term “dilution” here refers to a reduced dose of a protein solution, particularly a protein solution containing active substance.

The term “carrier gas” denotes a gas which absorbs a substance or a material and removes it from the process.

### EMBODIMENTS ACCORDING TO THE INVENTION

The present invention relates to an apparatus consisting of the following parts: (i) process chamber (7), (ii) vacuum pump (8), (iii) gas connector (1), (iv) at least 1 inlet (5), (v) at least 1 outlet (6), (vi) flow sensor (3), (vii) pressure sensor (4), (viii) at least 2 valves (2), (ix) optionally a perforated plate (10), (x) optionally a circulating pump (11) for protein solution (9) and a bypass with connectors (12) and (13). Embodiments by way of example are shown in FIGS. 1, 2 and 3. FIG. 1 (with the perforated plate (10)) shows by way of example an apparatus in which individual containers have preferably been placed. FIG. 2 shows by way of example an apparatus into which the protein solution (9) is introduced directly. FIG. 3 shows by way of example another apparatus which contains the protein solution (9) directly, the protein solution (9) in the embodiment shown being recirculated within the apparatus by means of a circulating pump (11) and the connectors (12 and 13).

The present invention relates to a method for concentrating a protein solution, comprising the following steps:
a. Preparing a protein solution,
b. Optionally transferring the protein solution from (a) into individual containers,
c. Transferring the protein solution from (a) or the individual containers from (b) into an apparatus comprising the following components:
i. process chamber (7),
ii. vacuum pump (8),
iii. gas connection (1),
iv. at least one inlet (5),
v. at least one outlet (6),
vi. flow sensor (3),
vii. pressure sensor (4),
viii. at least two valves (2),
ix. optionally a perforated plate (10),
x. optionally a recirculating pump (11) for protein solution (9) and bypass with connections (12) and (13),
d. Applying a gas current or carrier gas current to the apparatus from step c), wherein
xi. the process pressure is reduced,
xii. the flow rate of the gas or carrier gas is uniform,
e. Removing the concentrated protein solution or the individual containers from the apparatus according to method step c).

In a preferred embodiment of the method according to the invention the protein solution does not contain any adjuvants. The solvent for the protein is preferably water, e.g. WFI (water for injection) or G-water (=purified water).

In another embodiment of the method according to the invention the process pressure in is step d)i) is in the range from 10-600 mbar, 10-400 mbar, 10-200 mbar, preferably in the range from 10-100 mbar, and particularly preferably the process pressure is 100 mbar. At a process pressure below 10 mbar there is a danger of freezing.

In an exemplifying embodiment of the method according to the invention the temperature of the (carrier) gas is from 25° C. to 100° C., 25° C. to 40° C., preferably 40° C. or ambient temperature. The temperature of the (carrier) gas is preferably 40° C.

In another embodiment of the method according to the invention the process pressure in step d)i) is in the range from 10-100 mbar and the temperature of the (carrier) gas is 40° C. In an exemplifying embodiment of the method according to the invention the (carrier) gas is air, clean air, nitrogen, helium or argon, and preferably the (carrier) gas is dry air, preferably clean air, with a residual moisture content of less than 10% r.h. (relative humidity), less than 5% r.h., less than 1% r.h.

In another embodiment by way of example of the method according to the invention the flow speed is 4-7 L/min for a process chamber volume of 2.5 L to 3.5 L (=2.5-3.5 dm³). Preferably the temperature of the (carrier) gas in this embodiment is 25° C. to 40° C., most preferably 25° C.

A preferred embodiment of the method according to the invention is the concentration in 2R vials with an initial volume of up to 2 ml. This embodiment is further characterised in that the concentration is carried out at an absolute pressure of 100 mbar and a gas rate of 1.4-2.4 L/(min×dm³) standardised to the process chamber volume. Accordingly, the absolute gas rate for a chamber volume of 2.9 dm³(=L) is 4-7 L/min for example. The temperature of the gas at the inlet to the process chamber is about 40° C.

In another embodiment, protein solutions are transferred into 10R vials in a volume of up to 10 ml and then concentrated at a pressure of 100 mbar, a gas rate of 1.2-2.4 L/(min×dm³) standardised to the process chamber volume and a gas temperature of 40° C. at the inlet of the process chamber.

The method according to the invention operates in particular without losses, i.e. the active substance recovery is ≧95%. The method according to the invention operates without losses within the range of analytical accuracy (100%).

In another embodiment of the method according to the invention the protein solution contains an active substance, preferably a therapeutic active substance, preferably an is antibody. This also includes antibody fragments in principle. An antibody of type IgG1 is preferred.

In another embodiment of the method according to the invention the concentration of the protein solution in step (a) of the method according to the invention is 1-50 mg/ml, 20-50 mg/ml, 20-30 mg/ml, while the final concentration of the protein solution in step (e) of the method according to the invention is 5-400 mg/ml, 40-200 mg/ml, 80-200 mg/ml, preferably 100-200 mg/ml. An initial concentration of 10 mg/ml is also preferred.

In another embodiment of the method according to the invention the final concentration of the protein solution in step (e) of the method according to the invention is more than 50 mg/ml, more than 65 mg/ml, more than 80 mg/ml, more than 100 mg/ml, more than 200 mg/ml.

In another embodiment the concentration of the protein solution in step (a) of the method according to the invention is less than 50 mg/ml and the final concentration of the protein solution in step (e) of the method according to the invention is more than 50 mg/ml, more than 65 mg/ml, more than 80 mg/ml, more than 100 mg/ml, more than 200 mg/ml. Preferably, the protein is an antibody and the starting concentration is less than 50 mg/ml (preferably 10 mg/ml) and the final concentration is between 100-200 mg/ml.

In another embodiment of the method according to the invention re-buffering takes place between step (a) and (b) of the method, preferably re-buffering into an adjuvant-free solution such as water, for example WFI.

In another embodiment of the method according to the invention the concentrated protein solution is diluted after step (e). This dilution is carried out with water, for example. The concentration protein solution is diluted with a buffer or adjuvant solution, according to another embodiment. Preferably, an isotonic protein solution is produced from the concentration protein solution by diluting with buffer or adjuvant solution.

In another embodiment of the method according to the invention the protein solution that is to be concentrated is formulated in an adjuvant solution, the adjuvant concentration of which is in a reciprocal ratio to the concentration factor. If, for example, the protein solution is to be concentrated by a factor of 10, the concentration of the adjuvants at the start of the concentration process is 1/10 of the target concentration of the adjuvants.

In another embodiment of the method according to the invention concentration is carried out by a factor of 1.3 to 30 or 1.3 to 20, preferably by a factor of 10 to 20.

In another embodiment of the method according to the invention the protein solution in step (a) or (b) has a volume of less than 10 ml, between 2-8 ml, less than 1 ml. In another embodiment of the method according to the invention the protein solution in step (a) or (b) has a volume of less than 200 μl or 100 μl.

In another embodiment of the method according to the invention the method is carried out aseptically using a sterile filtered (carrier) gas such as clean air, for example.

The present invention relates to a method for measuring a protein concentration in a protein solution comprising the following steps:
a. preparing a protein solution with a defined protein concentration c_{A} in an individual container, the protein solution optionally being free from adjuvants,
b. measuring the weight m_{B} of the protein solution including individual container,
c. transferring the protein solution in the individual container from (b) into an apparatus comprising the following components:
   i. process chamber (7),
   ii. vacuum pump (8),
   iii. gas connection (1),
   iv. at least one inlet (5),
   v. at least one outlet (6),
   vi. flow sensor (3),
   vii. pressure sensor (4),
   viii. at least two valves (2),
   ix. optionally a perforated plate (10),
   x. optionally a recirculating pump (11) for protein solution (9) and bypass with connections (12) and (13),
d. applying a (carrier) gas current to the apparatus from step c), wherein
   i. the process pressure is reduced,
   ii. the flow rate of the (carrier) gas is uniform,
e. removing the concentrated protein solution in the individual container from the apparatus according to method step c).
f. measuring the weight m_{F} of the protein solution including individual container,
g. determining the quotient of the measurements obtained from method steps (b) and (f): m_{B}/m_{F},
h. calculating the protein concentration of the concentrated protein solution for example according to the following equation: C_{H}=m_{B}/m_{F}*c_{A}

The present invention further relates to a method for testing protein formulations comprising the following steps:
a. preparing a protein solution, the protein solution being free from adjuvants,
b. concentrating the protein solution from step a) using the method according to the invention, in which the solution is concentrated for example by a factor of 1.3 to 2.0,
c. re-diluting the protein solution using multiply concentrated buffer or adjuvant solution,
d. optionally setting a desired target concentration of the protein solution with water, for example WFI,
e. optionally testing the physico-chemical properties of the protein formulations and the protein integrity,
f. optionally selecting a (specified) protein formulation.

In an embodiment by way of example, at least 5, 10, 20, 50, 100 individual containers are prepared in step a) (high throughput method). For example, microtitre plates are also used.

The invention is hereinafter described in more detail by means of non-restrictive embodiments by way of example.

### EXAMPLES

### Example 1

### Determining the Evaporation Rates of Protein-Containing Solutions

Two IgG1 antibodies (IgG1a/IgG1b) and an IgG2 antibody (IgG2b) were re-buffered against water and then evaporated down under controlled conditions. The starting solutions of the monoclonal antibodies IgG1a and IgG2b were prepared by tangential flow filtration. The starting concentrations were 51 mg/mL for IgG1a and 87 mg/mL for IgG2b. The monoclonal antibody IgG1b was dialysed against water and adjusted to a protein concentration of 5.1 mg/ml.

The water evaporation rate was determined with a dynamic sorption balance (DVS made by SMS). This analytical device essentially consists of a sensitive balance with a sample crucible and a reference crucible (cf. schematic Figure). A defined gas current flows over the two crucibles at a defined relative humidity. A relative humidity of 0% r.h. was selected for the tests carried out. 100 μL aliquots of the protein solution were transferred into the sample crucible and dried in an air current of 200 cm³/min. The process temperature was 25° C.

FIGS. 4 to 6 show the changes in mass over the drying time.

The drying patterns are highly comparable for the 3 protein solutions used. The reduction in mass is substantially linear over a wide range. The evaporation rate does not collapse until just before the end of the concentration process, represented by a constant mass. No precipitates were formed during the drying of the adjuvant-free protein solutions, but instead transparent brittle films were produced at the end of the concentration process.

This Example shows that protein solutions dry comparably and uniformly, independently of the protein concentration and the protein used. This is an important condition for controlled concentration in order to prepare highly concentrated protein solutions.

### Example 2

### Air Drying of Adjuvant-Free Protein Solutions

In this Example the protein stability after carrier gas drying was investigated. For this purpose, the protein solutions containing buffer were re-buffered against water by tangential flow filtration and set to a starting concentration of 90-100 mg/ml. The concentration processes were carried out at atmospheric pressure, ambient temperature and a carrier gas rate of 30 L/min. Air free from water vapour was used as the carrier gas. The volume of the process chamber was about 32.5 dm³. The structure of the chamber corresponded to that in FIG. 1. For the concentration process, 100 μL portions of the protein solutions were placed in test tubes (internal diameter 5.0 mm).

Table 1 lists the antibodies used. The proteins IgG1c and IgG1d were monoclonal antibodies of type IgG1. The protein IgG2b was a monoclonal antibody of type IgG2.

By air drying it was possible to obtain protein concentrations of 320-402 mg/ml after 56 hours of process time. In spite of the absence of stabilisers the proteins exhibited only insignificant changes in their monomer content (FIG. 7).

To summarise, this Example shows that carrier gas drying is a very gentle process, which enables even adjuvant-free protein solutions to be concentrated without any substantial damage.

TABLE 1

| | initial | concentration after |
|---|---|---|
| | concentration, | 56 hours process |
| Protein | mg/ml | time, mg/ml |
| IgG1c | 90.1 | 320 |
| IgG1d | 102.0 | 402 |
| IgG2b | 91.8 | 329 |

### Example 3

In this Example the water evaporation rates were determined at different carrier gas rates and process pressures. 45 test tubes were each filled with 100 μL of water. The internal diameter of the test tubes was 5.0 mm. The resulting evaporation surface area was 78.5 mm². The process chamber had a volume of 2.9 dm³. The process time was 10 hours. FIG. 8 shows the drying patterns under the different process conditions. The reductions in mass showed a well approximated linear pattern. The evaporation rates obtained from the regression are shown in Table 2. It is apparent that acceptable evaporation rates are obtained only when reduced pressure and carrier gas are used simultaneously. The use of carrier gas (tests numbers 2-4) gave a more homogeneous evaporation of water, compared with the test without carrier gas (test number 1)—expressed as the relative standard deviation over the losses of mass of the individual containers after 10 hours.

TABLE 2
Evaporation rates

| Test | Process pressure, | Carrier gas | Evaporation | Relative standard |
|---|---|---|---|---|
| No. | mbar | rate, L/min | rate, mg/h | deviation, % |
| 1 | 600 | 0 | 0.8 | 8 |
| 2 | 1013 | 4 | 2.6 | 3 |
| 3 | 600 | 4 | 4.2 | 3 |
| 4 | 200 | 4 | 9.4 | 4 |

### Example 4

An IgG4 antibody solution (IgG4a) was re-buffered against WFI (Water for Injection) by tangential flow filtration and adjusted to a starting concentration of 27 mg/ml. A volume of 2 ml of this protein solution was placed in a 2R vial with an internal diameter of 14 mm, corresponding to an exchange surface of 1.54 cm², and concentrated by carrier gas drying. The test set-up corresponded to that in FIG. 1. The process chamber had a volume of 2.9 dm³. The concentration process was carried out at a process pressure of 100 mbar and a carrier gas rate of 7 L/min. The carrier gas was heated to 40° C. In this is way the temperature in the chamber was set to 26-27° C. Air free from water vapour was used as the carrier gas. FIG. 9 shows the quantities of water evaporated and the corresponding protein concentrations plotted against the process time. The water evaporation rate was 50 mg/ml. The evaporation rate was constant up to the end of the process until a protein concentration of 235 mg/ml was achieved. Table 3 lists the monomer contents and viscosities that correspond to the protein concentration. The monomer content does not show any changes over the process time. The achievable viscosities of 357 mPas particularly indicate the technical advantage of this process, as there was no deterioration in the rate of concentration despite this very high viscosity.

TABLE 3

| Protein concentration, | Monomer content, | Viscosity, |
|---|---|---|
| mg/ml | % | mPas |
| 27 | 99 | 1.63 |
| 68 | 99 | 6.32 |
| 95 | 99 | 9.77 |
| 115 | 99 | 16.8 |
| 142 | 99 | — |
| 235 | 99 | 357 |

### Example 5

An IgG4 antibody solution (IgG4a) was re-buffered by tangential flow filtration against WFI (Water for Injection) and adjusted to a starting concentration of 27 mg/ml. The test set-up corresponded to that in FIG. 1. The process chamber had a volume of 2.9 dm³. The concentration was carried out at a process pressure of 100 mbar and a carrier gas rate of 7 L/min. The temperature in the chamber was adjusted to 26-27° C. by heating the carrier gas. Air free from water vapour was used as the carrier gas. The concentration was carried out in 2 ml vials with an internal diameter of 14 mm and an exchange surface of 1.54 cm².

The protein solution to be concentrated was added to the vial, distributed over 9 individual additions over the process time. The protein solution remaining in each case was stored at 5° C.±3° C. Table 4 shows the amounts added at the corresponding process times. In all, a quantity of 1.46 g of protein solution was added. 10 vials were stored and taken out at different process times. FIG. 10 shows the protein concentrations at different points in time. The protein concentrations determined gravimetrically were compared with the protein concentrations determined by UV spectroscopy. It can be inferred from the good conformity between the protein concentration determined gravimetrically and that determined by UV spectroscopy that the concentration process takes place without any significant protein losses.

Table 5 shows the monomer contents as a function of the protein concentration. No changes can be detected.

TABLE 4

| | Process time, min | Amount added, mg |
|---|---|---|
| Start | 0 | 235 |
| addition 1 | 120 | 95 |
| addition 2 | 240 | 90 |
| addition 3 | 425 | 138 |
| addition 4 | 605 | 239 |
| addition 5 | 775 | 144 |
| addition 6 | 955 | 145 |
| addition 7 | 1150 | 136 |
| addition 8 | 1340 | 143 |
| addition 9 | 1570 | 94 |

TABLE 5

| | Protein concentration, | Monomer content, |
|---|---|---|
| Sample | mg/ml | % |
| sample 1 | 94 | 99 |
| sample 2 | 136 | 99 |
| sample 3 | 178 | 99 |
| sample 4 | 201 | 99 |
| sample 5 | 302 | 99 |

As can be seen from the Example, concentration may be carried out by sequential addition of the protein solution. It is also demonstrated that the concentration process does not result in the loss of any protein.

### Example 6

An adjuvant-free IgG1 antibody (IgG1d) solution with a protein concentration of 50 mg/ml was concentrated to about 140 mg/ml by carrier gas drying and then re-diluted with multiply concentrated adjuvant solutions to a protein concentration of 90 mg/ml. For this, 1 ml of the IgG1d solution was transferred into 2R vials and placed in the process chamber. The test set-up corresponded to that in FIG. 1. The chamber volume was 2.9 dm³. The concentration process was carried out at a process pressure of 100 mbar and a carrier gas rate of 7 L/min. The carrier gas used was air free from water vapour at a temperature of 40° C. The temperature in the process chamber was 26-27° C. The protein concentrates were diluted accordingly, as described in Table 9, and different formulations were prepared using this dilution. The protein concentrations were determined gravimetrically from the initial concentration and the weight of water lost during concentration. Four times concentrated adjuvant solutions and WFI (Water for Injection) were used for the dilution. The compositions of the finished diluted formulations are shown in Table 7. The protein concentrations were determined by UV spectroscopy. The protein concentrations measured accorded very well with the target concentration to be set.

TABLE 6
Dilution matrix

| Vial Nr. | 1 | 9 | 2 | 3 | 4 | 5 | 6 | 7 | 8 |
|---|---|---|---|---|---|---|---|---|---|
| protein conc. after | 155 | 141 | 141 | 150 | 142 | 141 | 159 | 139 | 146 |
| concentration | | | | | | | | | |
| process, mg/ml | | | | | | | | | |
| target concentration, | 90 | 90 | 90 | 90 | 90 | 90 | 90 | 90 | 90 |
| mg/ml | | | | | | | | | |
| volume of protein | 250 | 250 | 250 | 250 | 250 | 250 | 250 | 250 | 250 |
| concentrate, μL | | | | | | | | | |
| volume of adjuvant | — | 98 | 98 | 104 | 98 | 98 | 110 | 97 | 101 |
| solution (four times | | | | | | | | | |
| concentrated), μL | | | | | | | | | |
| vol of WFI, μL | | 44 | 43 | 63 | 45 | 44 | 81 | 40 | 54 |
| total volume, μL | 431 | 393 | 391 | 418 | 394 | 392 | 441 | 386 | 406 |

TABLE 7

| | protein concentration, | | |
|---|---|---|---|
| | mg/ml | adjuvant composition | pH |
| formulation 1 | 87 | — | not set |
| formulation 2 | 91 | 25 mM succinate/ | 6.5 |
| | | 0.02% w/v Tween 20 | |
| formulation 3 | 89 | 25 mM succinate/ | 6.5 |
| | | 210 mM saccharose | |
| | | 0.02% w/v Tween 20 | |
| formulation 4 | 93 | 25 mM succinate/ | 6.5 |
| | | 210 mM sorbitol | |
| | | 0.02% w/v Tween 20 | |
| formulation 5 | 92 | 25 mM succinate/ | 6.5 |
| | | 125 mM NaCl | |
| | | 0.02% w/v Tween 20 | |
| formulation 6 | 91 | 25 mM citrate | 6.0 |
| | | 0.02% (v/w) Tween 20 | |
| formulation 7 | 87 | 25 mM citrate | 6.0 |
| | | 210 mM saccharose | |
| | | 0.02% (v/w) Tween 20 | |
| formulation 8 | 88 | 25 mM citrate | 6.0 |
| | | 215 mM sorbitol | |
| | | 0.02% (v/w) Tween 20 | |
| formulation 9 | 90 | 25 mm citrate | 6.0 |
| | | 125 mM NaCl | |
| | | 0.02% (v/w) Tween 20 | |

This Example demonstrates the suitability of this process for preparing highly concentrated protein solutions by rediluting adjuvant-free protein solutions with multiply concentrated adjuvant concentrates. The measured protein concentrations agreed very closely with the target concentrations. By adding the adjuvants directly, undesirable fluctuations in the adjuvants can be avoided. These occur particularly in manufacturing processes that adjust the formulations through semipermeable membranes, such as for example in tangential flow filtration. This Example also shows that it is possible to carry out formulation screening with minimal amounts. In this Example the concentration process was carried out with an initial volume of protein solution of only 1 ml of each formulation to be tested. 250 μL of protein solution were used for the redilution.

### Example 7

5 ml of a solution consisting of a FAB antibody fragment with a mass of 48 kDa and a is buffer system consisting of 1 mM citrate, 1 mM phosphate and 1 mM acetate with a pH of 6.5 were transferred into a 10R vial by three individual additions. The internal diameter of the vial was 22 mm, corresponding to an exchange surface of 380 mm². The initial concentration was 1.9 mg/ml. The vial was transferred into a process chamber with a volume of 2.9 dm³ and the solution was concentrated at 100 mbar and at a carrier gas rate of 7 L/min. The carrier gas used was air free from water vapour. The set-up corresponded to that in FIG. 1. The process was run without any temperature control of the carrier gas. The final concentration achieved was 26 mg/ml. In this process, once stable process conditions had been established, an average water evaporation rate of 105 mg/h was achieved.

1) A method for concentrating a protein solution, comprising the following steps:
a. preparing a protein solution,
b. optionally transferring the protein solution from (a) into individual containers,
c. transferring the protein solution from (a) or the individual containers from (b) into an apparatus comprising the following components:
i. process chamber (7),
ii. vacuum pump (8),
iii. gas connection (1),
iv. at least one inlet (5),
v. at least one outlet (6),
vi. flow sensor (3),
vii. pressure sensor (4),
viii. at least two valves (2),
ix. optionally a perforated plate (10),
x. optionally a recirculating pump (11) for protein solution (9) and bypass with connections (12) and (13),
d. applying a carrier gas current to the apparatus from step c), wherein
i. the process pressure is reduced,
ii. the flow rate of the carrier gas is uniform,
e. removing the concentrated protein solution or the individual containers from the apparatus according to method step c).

2) The method according to claim 1, wherein the protein solution does not contain any adjuvants.

3) The method according to claim 2, wherein the solvent for the protein is water, for example WFI (water for injection).

4) The method according to claim 1, wherein the process pressure in step d)i) is in the range from 10-600 mbar, 10-400 mbar, 10-200 mbar, preferably in the range from 10-100 mbar, and most preferably the process pressure is 100 mbar.

5) The method according to claim 1, wherein the temperature of the carrier gas is 25° C. to 100° C., 25° C. to 40° C., preferably 40° C. or ambient temperature.

6) The method according to claim 1, wherein the process pressure in step d)i) is in is the range from 10-100 mbar and the temperature of the carrier gas is 40° C.

7) The method according to claim 1, wherein the carrier gas is air, clean air, nitrogen, helium or argon, and the carrier gas is preferably dry air, preferably clear air, with a residual moisture content of less than 10% r.h. (relative humidity), less than 5% r.h., less than 1% r.h.

8) The method according to claim 1, wherein the flow rate is 4 to 7 L/min.

9) The method according to claim 1, wherein the method operates without any losses.

10) The method according to claim 1, wherein the protein solution contains an antibody.

11) The method according to claim 1, wherein the final concentration of the protein solution in step (e) of claim 1 is more than 50 mg/ml, more than 65 mg/ml, more than 80 mg/ml, more than 100 mg/ml, more than 200 mg/ml.

12) The method according to claim 1, wherein between steps (a) and (b) re-buffering takes place, preferably re-buffering into an adjuvant-free solution such as water, for example WFI.

13) The method according to claim 1, wherein the concentrated protein solution is diluted after step (e).

14) The method according to claim 1, wherein the concentrated protein solution is diluted with a buffer or adjuvant solution, preferably an isotonic protein solution is prepared from the concentrated protein solution by dilution with buffer or adjuvant solution.

15) The method according to claim 1, wherein concentration by a factor of 1.3 to 30 or 1.3 to 20 is carried out, preferably by a factor of 10 to 20.

16) The method according to claim 1, wherein the protein solution in step (a) or (b) has a volume of less than 10 ml, between 2 and 8 ml, less than 1 ml.

17) The method according to claim 1, wherein the method is carried out aseptically using a sterile-filtered carrier gas such as clean air, for example.

18) A method for measuring a protein concentration in a protein solution comprising the following steps:
a) preparing a protein solution with a defined protein concentration c_{A} in an individual container, the protein solution optionally being free from adjuvants,
b) measuring the weight m_{B} of the protein solution including individual container,
c) transferring the protein solution in the individual container from (b) into an apparatus comprising the following components:
i. process chamber (7),
ii. vacuum pump (8),
iii. gas connection (1),
iv. at least one inlet (5),
v. at least one outlet (6),
vi. flow sensor (3),
vii. pressure sensor (4),
viii. at least two valves (2),
ix. optionally a perforated plate (10),
x. optionally a recirculating pump (11) for protein solution (9) and bypass with connections (12) and (13),
d) applying a carrier gas current to the apparatus from step c), wherein
i. the process pressure is reduced,
ii. the flow rate of the carrier gas is uniform,
e) removing the concentrated protein solution in the individual container from the apparatus according to method step c),
f) measuring the weight m_{F} of the protein solution including individual containers,
g) determining the quotient of the measurements obtained from method steps (b) and (f): mₑ/m_{F},
h) calculating the protein concentration of the concentrated protein solution for example according to the following equation: c_{H}=m_{B}/m_{F}*c_{A}

19) A method for testing protein formulations comprising the following steps:
a) preparing a protein solution, the protein solution being free from adjuvants,
b) concentrating the protein solution from step a) using the method according to claim 1, the solution being concentrated for example by a factor of 1.3 to 2.0,
c) re-diluting the protein solution using multiply concentrated buffer or adjuvant solution,
d) optionally setting a desired target concentration of the protein solution with water, for example WFI,
e) optionally testing the physico-chemical properties of the protein formulations and the protein integrity,
f) optionally selecting a protein formulation.
